(19) United States
(12) Patent Application Publication
(10) Pub. No.: US 2008/0108824 A1
(43) Pub. Date: May 8, 2008

(54) Medical Devices Incorporating a Bioactive and Methods of Preparing Such Devices

(75) Inventors: Andrew P. Isch, Lafayette, IN (US); Patrick H. Ruane, Hayward, CA (US); Waleska Perez-Segarra, Lafayette, IN (US)

Correspondence Address: BRINKS HOFER GILSON & LIONE/CHICAGO/COOK, PO BOX 10395, CHICAGO, IL, 60610, US

(73) Assignee: MED Institute, Inc, West Lafayette, IN (US)

(21) Appl. No.: 11/862,925

(22) Filed: Sep. 27, 2007

Related U.S. Application Data
(60) Provisional application No. 60/847,857, filed on Sep. 28, 2006.

(51) Int. Cl. C07D 211/08; A61F 2/02

(52) U.S. Cl. 546/323; 623/11.11

(57) ABSTRACT

One embodiment provides a medical device comprising a base material and a bioactive in contact with the base material, the bioactive having a proton binding site with a non-ionic form and an ionic form, the bioactive being less soluble in water when the proton binding site is in the non-ionic form than when the proton binding site is in the ionic form, wherein at least 5% w/w of the bioactive is present with the proton binding site in the non-ionic form and wherein the bioactive is not an anesthetic. Another embodiment provides such a medical device where the bioactive is an anesthetic and where the device is not a ureteral stent. Another aspect provides method of manufacturing such devices.

### RELATED APPLICATIONS

This application claims the benefit of the filing date under 35 U.S.C. §119(e) of Provisional U.S. Patent Application Ser. No. 60/847,857, filed Sep. 28, 2006, the contents of which are hereby incorporated by reference.

### TECHNICAL FIELD

The technical field of the patent is that of medical devices containing a bioactive and processes for incorporating a bioactive with such devices.

### BACKGROUND

It has become common to treat a variety of medical conditions by introducing an implantable medical device partly or completely into the esophagus, trachea, colon, biliary tract, urinary tract, vascular system or other location within a human or veterinary patient. For example, many treatments of the vascular system entail the introduction of a device such as a stent, a catheter, a balloon, a wire guide, a cannula, or the like.

For certain applications, the medical device is coated with a bioactive adapted to expose tissue within the body to the bioactive over a desired time interval, such as by releasing the bioactive. Desirably, the bioactive is released within the body at a reproducible and predictable fashion so as to optimize the benefit of the bioactive to the patient over the desired period of time. Providing coated medical devices adapted to release a bioactive at a desired rate over a period of time is one challenge in designing implantable medical devices. For example, a coated medical device may release a bioactive at a greater rate than desired upon implantation, and subsequently release the bioactive at a slower rate than desired at some time after implantation. What is needed is a medical device that provides for release of one or more bioactives over a period of time that is desired for one or more bioactive applications, desirably at an optimal elution rate from the device.

Many bioactives contain a proton binding site and are either insoluble or sparingly soluble in an aqueous environment when the proton binding site is in a non-ionic form. Because of this limited solubility, such drugs are often provided in a more soluble ionic analog or form, such as a hydrochloride or sodium salt. However bioactives in the salt form may elute from a medical device too quickly for medical applications where it may be more desirable to retain the bioactive in the coating for a longer period of time. For example, anesthetic bioactive coatings are preferably retained on a surface of a medical device for implantation within the urinary tract. Anesthetic bioactive compounds having an ionic and non-ionic form include 1-piperidine-2-carboxanilide local anesthetic bioactives, including compounds comprising the chemical structure of formula 1: 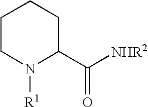
where R¹ is methyl (mepivacaine), n-propyl (ropivacaine as the S-enantiomer) or n-butyl (bupivacaine) and R² is 2,6-dimethylphenyl. For example, implantation of a medical device coated with an ionic form of the anesthetic drug Bupivacaine (1-butyl-N-(2,6-dimethylphenyl)-2-piperidinecarboxamide hydrochloride) (i.e. Bupivacaine HCl) may result in the majority of the drug being released into body fluids before the device reaches the desired implantation site. Moreover, coating salt forms of such anesthetics on medical devices such as catheters or ureteral stents may result in a coating that is too soluble upon implantation, reducing the period of time during which the anesthetic is present in the coating.

### SUMMARY

The present disclosure relates to medical devices incorporating at least one bioactive and having a desired rate of bioactive release and methods of manufacturing such medical devices. A bioactive may have pH-dependent solubilities in different solvents, such as water or an alcohol (e.g., methanol, ethanol, etc). The difference in solubility of the bioactive may depend on the pKa of an acidic proton in the bioactive. Increasing the pH of the solution containing the bioactive above the pKa may decrease the solubility of the bioactive in an aqueous solvent, but increase the solubility of the bioactive in other solvents, such as an alcohol. The bioactive may be incorporated into a medical device by contacting a solution containing the bioactive in a solvent at a controlled pH and removing the solvent to form the device containing the bioactive.

Preferably, the bioactive includes an acidic proton providing the bioactive with a pH dependent solubility. In addition, the bioactive is preferably applied to a medical device by applying a solution having the bioactive in a solvent at a controlled pH that is at least one pH unit above the pKa of an acidic proton in the bioactive. The medical device can be removed from contact with the solvent, to form a device incorporating the bioactive and having a desired elution rate of release of the bioactive in an aqueous solution.

The proton binding site may have an ionic form and non-ionic form depending on the pH of a coating solution. For example, the proton binding site may comprise a nitrogen atom providing a cationic protonated quaternary amine form and a non-ionic deprotonated tertiary amine form, with the bioactive having greater water solubility when the proton binding site is in the ionic form. Preferred devices include such a bioactive with the proton binding site in the form providing the lower water solubility to the bioactive. The water solubility of the bioactive may differ when the proton binding site is in the ionic form or the non-ionic form.

In one embodiment, the bioactive is incorporated into a base material forming at least a portion of the device, for example, into pores formed in a polymeric base material. In another embodiment, the bioactive is coated onto a surface of the base material. The solubility of the resulting bioactive portion of the device, and therefore the rate of elution of the bioactive upon implantation, may be altered by changing the pH of the solution.

Preferably, the medical device comprises of a bioactive with an acidic proton binding site, where the acidic proton is characterized by a pKa. The medical device incorporating the bioactive may be formed by contacting a solution including a solvent, such as an alcohol, with the bioactive at a pH greater than the pKa of the acidic proton and removing the solvent from the solution to form a medical device incorporating the bioactive. Increasing the pH of the solution above the pKa increases the fraction of the bioactive molecules in the resulting coating with the proton binding site in the unprotonated form. In one embodiment, the pH in the solution is at least one pH unit above the pKa of the acidic proton of the proton binding site. In another embodiment, the pH in the solution is at least two pH units above the pKa of the acidic proton of the proton binding site. Since the bioactive having the proton binding site in the protonated form may have a different solubility than the bioactive with the proton binding site in the unprotonated form, the elution rate of the bioactive may differ depending on the pH of the coating solution. Therefore, the rate of release of the bioactive from the device may be changed by altering the pH of the coating solution.

In one embodiment, the bioactive is an anesthetic with a nitrogen-containing proton binding site, such as bupivacaine. Bupivacaine has a proton binding site having an acidic proton pKa of about 8. The proton binding site in bupivacaine is a tertiary amine in the non-ionic, unprotonated form and a cationic quatranary amine in the protonated ionic form. Bupivicaine has a higher solubility in water when the proton binding site is in the ionic, protonated form than when the proton binding site is in the non-ionic, deprotonated form. By increasing the pH of a solution above the pKa of the proton binding site, more of the bupivicaine with the proton binding site in the unprotonated non-ionic form is incorporated into the device, and the solubility of a resulting bupivicaine portion of the device decreases in water. Therefore, the rate of elution of bupivicaine in an aqueous environment (e.g., within the urinary tract) may be prolonged by applying the coating as a solution containing a removable solvent and the bupivacaine at a pH greater than the pKa of the proton binding site of bupivicaine. In another embodiment, the bioactive is a compound other than an anesthetic.

In one aspect, the bioactive is present in a coating formed on a base material of the medical device. In various embodiments, the coating consists essentially of the bioactive. For example, a coating may consist essentially of the bioactive, with a desired percent weight of the coating consisting of the less water-soluble form of the bioactive with the proton binding site in the unprotonated non-ionic form. The remaining weight of the coating may consist of the more water-soluble bioactive with the proton binding site in the protonated ionic form. Optionally, the coating may also include other materials that do not affect the rate of release of the bioactive upon implantation of the coated medical device.

Optionally, the medical device coating may further comprise one or more materials selected to alter the rate of release of the bioactive from the coating, such as a biodegradable polymer mixed with or coated on top of the bioactive material. In certain embodiments, the coating layer includes a carrier, which may include a bioelastomer, PLGA, PLA, PEG, Zein (a protein extract obtained form corn), or a hydrogel. In certain other embodiments, the carrier includes microcrystalline cellulose, hydroxypropylmethyl cellulose, hydroxypropyl cellulose, a cellulose product, a cellulose derivative, a polysaccharide or a polysaccharide derivative. In other embodiments the carrier includes lactose, dextrose, mannitol, a derivative of lactose, dextrose, mannitol, starch or a starch derivative. Optionally, the coating layer further includes a second bioactive.

In other embodiments, the bioactive in present within a base material forming at least part of the structure of the medical device, for example, within pores formed in a polymeric base material. In such embodiments, a desired percent weight of the bioactive consists of the less water-soluble form of the bioactive with the proton binding site in the unprotonated non-ionic form. The remaining weight of the bioactive consists of the more water-soluble bioactive with the proton binding site in the protonated ionic form.

In another aspect, provides methods of incorporating a bioactive into a medical device. The bioactive preferably includes a proton binding site providing a pH dependent solubility in a solvent. The solubility of the bioactive in an aqueous solvent (e.g., water) may be higher when the proton binding site is in an ionic form than when the proton binding site is in a non-ionic form. Preferably, the proton binding site comprises nitrogen, such as a tertiary amine non-protonated non-ionic form and a quaternary amine cationic protonated form.

A method may include contacting a medical device with a solution containing a bioactive and a solvent at a controlled pH and removing the solvent to form a medical device comprising the bioactive. Preferably, the bioactive has a proton binding site for an acidic proton characterized by a pKa. A bioactive with a nitrogen-containing proton binding site may have a greater water-solubility when the proton binding site is in a protonated and ionic form than when the proton binding site is in a deprotonated and non-ionic form. Most preferably, the coating method includes the step of contacting the medical device with a solution at a pH greater than the pKa of the proton binding site of the bioactive. Increasing the pH of a solution more than one pH unit above the pKa of a nitrogen-containing proton binding site may substantially decrease the rate at which the incorporated bioactive elutes from the medical device in an aqueous environment.

In another aspect, the methods of incorporation may include (1) dissolving the bioactive in a solvent (e.g., water) with the proton binding site in the ionic form to form a first solution; (2) adjusting the pH of the first solution to convert at least a fraction of proton binding sites to the non-ionic form, where the bioactive is less soluble in the solvent when the proton binding site in the non-ionic form than in the ionic form; (3) isolating the fraction of the bioactive with the proton binding site in the non-ionic form from the first solution; (4) dissolving the bioactive with the proton binding site in the non-ionic form in a second solvent to form a solution and (5) incorporating the bioactive in the solution into a medical device to form a medical device comprising the bioactive having the proton binding site in the non-ionic form. For example, the bioactive bupivacaine HCl having a quaternary (protonated) amine cationic proton binding site may be dissolved in water to form a clear first solution at a neutral pH (e.g., about pH 7). The pH of the solution may be lowered until bupivacaine with the proton binding site in the non-ionic deprotonated form precipitates as a white solid out of the first solution. The solid bupivacaine precipate may be isolated from the first solution and dissolved in a suitable organic solvent, such as dichloromethane, and spray coated onto the surface of a medical device to form a bupivacaine coating. The resulting bupivacaine coating may have a reduced rate of elution upon implantation within the urinary tract compared to a coating consisting of bupivacaine HCl.

### BRIEF DESCRIPTION OF THE DRAWINGS

FIGS. 1-5A depict medical devices useful in coated embodiments;

FIG. 6A is a partial, enlarged top view of a portion of an endolumenal medical device. FIGS. 6B-6F are enlarged cross-sectional views along lines B-B′ of the endolumenal medical device of FIG. 6A;

FIG. 7 is a graph depicting elution profiles of a coated stent embodiment. The graph shows the elution of bupivacaine in buffers having pH values of 4.5 (●), 7.0 (▪) and 9.5 (▴) over a 12-day period.

FIGS. 8-9 are illustrations depicting coatings of prior art pharmacologically active substances onto stents; and

FIGS. 10-11 are illustration depicting coating of non-ionic pharmacologically active substances onto stents.

FIG. 12 is a graph showing the elution of bupivacaine from coated polyurethane drainage stents.

### DETAILED DESCRIPTION

### Definitions

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of” and “consisting essentially of,” the embodiments or elements presented herein, whether explicitly set forth or not.

The terms “about” or “substantially” used with reference to a quantity includes variations in the recited quantity that are equivalent to the quantity recited, such as an amount that is insubstantially different from a recited quantity for an intended purpose or function.

The term “alloy” refers to a substance composed of two or more metals or of a metal and a nonmetal intimately united, such as by chemical or physical interaction. Alloys can be formed by various methods, including being fused together and dissolving in each other when molten, although molten processing is not a requirement for a material to be within the scope of the term “alloy.” As understood in the art, an alloy will typically have physical or chemical properties that are different from its components.

As used herein, the term “implantable” refers to an ability of a medical device to be positioned, partially or wholly, at a location within a body of a human or veterinary patient for any suitable period of time, such as within a body vessel. Furthermore, the terms “implantation” and “implanted” refer to the positioning of a medical device, partially or wholly, at a location within a body, such as within a body vessel. Implantable medical devices can be configured for transient placement within a body vessel during a medical intervention (e.g., minutes to hours), or to remain in a body vessel for a prolonged period of time after an implantation procedure (e.g., weeks or months or years). Implantable medical devices can include devices configured for bioabsorption within a body during a prolonged period of time.

The term “biodegradable” refers to materials selected to dissipate upon implantation within a body, independent of which mechanisms by which dissipation can occur, such as dissolution, degradation, absorption and excretion. The actual choice of which type of materials to use may readily be made by one of ordinary skill in the art. Such materials are often referred to by different terms in the art, such as “bioresorbable,” “bioabsorbable,” or “biodegradable”, depending upon the mechanism by which the material dissipates. The prefix “bio” indicates that the erosion occurs under physiological conditions, as opposed to other erosion processes, caused for example, by high temperature, strong acids or bases, UV light or weather conditions.

The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause an undesirably adverse, long-lived or escalating biological reaction or response, and is distinguished from a mild, transient inflammation which typically accompanies surgery or implantation of foreign objects into a living organism.

A “non-bioabsorbable” or “biostable” material refers to a material, such as a polymer or copolymer, which remains in the body without substantial bioabsorption.

As used herein, the terms “bioactive” or “bioactive” refer to any pharmaceutically active agent that produces an intended therapeutic effect on the body to treat or prevent conditions or diseases.

As used herein, a “mixture” refers to a combination of two or more substances in which each substance retains its own chemical identity and properties.

As used herein, a “barrier layer” is any layer that is placed over at least a portion of a bioactive present in or on an implantable medical device. In general, the bioactive will not be present in the barrier layer. Any mixing of a bioactive with the barrier layer is unintentional and merely incidental. The barrier layer may or may not be the outer most layer present on the implantable medical device. For example, a bioactive may be coated onto a surface of a medical device, a first barrier layer placed over the bioactive and further barrier layers and layers contain the same or a different bioactive placed on the first barrier layer. The barrier layer may control the release of the bioactive from the medical device upon implantation.

As used herein, a “carrier material” refers to a material that forms a mixture with bioactive on or in an implantable medical device. The carrier material may control the release of the bioactive from the medical device.

Implantable Medical Devices Including a Bioactive

A first aspect provides an implantable medical device (“medical device”) including at least one bioactive having a proton binding site having an ionic form and a non-ionic form and providing a controlled release of the bioactive into the adjacent or surrounding tissue upon implantation of the medical device. References to “the proton binding site in the non-ionic form” and the “proton binding site in the non-ionic form” refer to one or more proton binding sites on the bioactive that provide different water solubilities to the bioactive in the ionic form and the non-ionic form.

Preferably, the bioactive is a compound having a greater water solubility when the proton binding site is in a protonated ionic form and a lower water solubility when the proton binding site is in a deprotonated non-ionic form. Medical devices including a bioactive with such a proton binding site may offer several advantages. For example, incorporating required portions of the ionic form and the non-ionic form of the bioactive, the medical device allows for amounts of the bioactive to be released for longer periods of time as compared to the release from previous devices.

In certain embodiments, the bioactive is present in a coating on a base material of the medical device. In various embodiments, the coating consists essentially of the bioactive. For example, a coating may consist essentially of the bioactive, with a desired percent weight of the coating consisting of the less water-soluble form of the bioactive with the proton binding site in the non-ionic form. The remaining weight of the coating may consist of the more water-soluble bioactive with the proton binding site in the ionic form. Optionally, the coating may also include other materials, including materials that do not affect the rate of release of the bioactive upon implantation of the medical device.

In other embodiments, the bioactive is contained within a base material forming at least part of the structure of the medical device, for example, within pores formed in a polymeric base material. In such embodiments, a desired percent weight of the bioactive consists of the less water-soluble form of the bioactive with the proton binding site in the non-ionic form. The remaining weight of the bioactive consists of the more water-soluble bioactive with the proton binding site in the ionic form.

The proton binding site may exist in a pH-dependent equilibrium in an aqueous environment between a protonated and unprotonated form. The ionic form of the proton binding site of such bioactives preferably contains an acidic proton bound to the proton binding site, such as an ionic protonated quaternary amine structure. For example, the bioactive may be a piperidine carboxanilide compound, such as a compounds comprising the chemical structure of formula (1) in an unprotonated and non-ionic form: 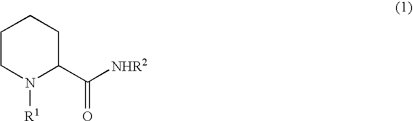
where R¹ is an alkyl group and R² is an optionally alkyl substituted aryl group. In formula (1), the proton binding site is preferably the tertiary amine bound to moiety R¹. In certain embodiments, the piperidine carboxanilide compound is an anesthetic, for example, a local anesthetic which causes loss of feeling in a part of the body without affecting consciousness.

Preferably, the medical device is manufactured having at least a portion of the bioactive present with the proton binding site in the non-ionic form. In one embodiment, at least 5% wt/wt of the bioactive in present in the non-ionic form. In other embodiments, at least 10%, 20%, 30%, 40%, 50%, 60%, 70%, 80%, 90%, 95%, 99% or 100% wt/wt of the bioactive is present in the non-ionic form. The amount of the bioactive present with the proton binding site in a non-ionic or ionic form may be determined by the pH of a coating solution containing the bioactive. In solution, the ionic form of the proton binding site may exist in pH dependent equilibrium with the non-ionic form in which the proton is absent from this site. The relationship between the percentage of the bioactive with the proton binding site in the ionic and non-ionic forms is given by the Henderson-Hasselbach equation:
pH=pKa+log([non-ionic form]/[ionic form])
where [ionic form] refers to the concentration of the bioactive molecule in the protonated state, and [non-ionic form] refers to the concentration of the bioactive molecule in the deprotonated state. Accordingly, when a bioactive with an acidic proton binding site is placed in a solution at a pH equal to the pKa of the acidic proton, 50% of the bioactive exists in the protonated state and about 50% is in the deprotonated state. In one embodiment, the “acidic proton binding site” can be any portion of a molecule (preferably including a nitrogen atom proton binding site) capable of covalently binding a proton (H⁺) that is released at a pH of between about −1.76 and 15.76, preferably between about 0 and 14, and more preferably between 4 and 10. Typically, the ionic form is more soluble in polar solvents such as water, while the non-ionic form can be more soluble in non-polar solvents. Frequently, bioactives comprising a proton binding site are prescribed or provided in an ionic form.

Preferably, the medical device includes the bioactive with a cationic deprotonated and nitrogen-containing proton binding site such that the bioactive has higher water solubility when the proton binding site is in the ionic form than in a deprotonated non-ionic form. Medical devices comprising the bioactive with a proton binding site in the non-ionic form may allow drug doses in the device to be minimized to those necessary for treatment. Administering lesser quantities of bioactives may minimize undesirable side effects of the bioactives. In addition, medical device containing the bioactive with the proton binding site in the non-ionic form may also be more durable than devices having the bioactive with the proton binding site in the ionic form.

A coating made with the bioactive having the proton binding site in the non-ionic form can be more adherent than a coating containing substantially the ionic form of the bioactive. In some cases, the bioactive having the proton binding site in the ionic form exists as a crystalline structure, resulting in a rough coating having poor adhesion properties, whereas the non-ionic form exists as a fine powder that forms a smoother coating having superior adhesion to the surface of the medical device. In addition, medical device coatings with the bioactive having the proton binding site in the non-ionic form may be well-suited for placement adjacent to or in contact with a lipophilic surface within a body (for example, a cellular surface having lipophilic cell membranes). Contacting such a surface with a coating of a medical device containing a bioactive with the proton binding site in a non-ionic form may enhance absorption of the bioactive into the cell by allowing the non-ionic form, which is often more lipophilic than the ionic form, to more easily cross the cell membrane and enter the cell before conversion to the active ionic form.

Bioactives having Ionic and Non-ionic Forms

The bioactive may include any suitable pharmaceutically active agent having a proton binding site and different water solubilities when the proton binding site is in an ionic form and a non-ionic form. The proton binding site is preferably unprotonated in the non-ionic form and protonated in the ionic form. The proton binding site is preferably cationic in the ionic form. The bioactive may have multiple proton binding sites providing different water solubility to the bioactive depending on whether each proton binding site is in the ionic or non-ionic form.

In one embodiment, the proton binding site may include a nitrogen atom, and may be a primary, secondary, or tertiary amine in a non-ionic unprotonated form. For example, the bioactive preferably comprises a chemical structure of formula (I) above, where R1 is selected from the group consisting of methyl (mepivacaine), n-propyl (ropivacaine as the S-enantiomer) or n-butyl (bupivacaine) and R² is 2,6-dimethylphenyl.

Another undesirable aspect of using the ionic form of the bioactive is that this form may be more hydrophilic than lipophilic. The non-ionic form may be more useful because it may be more easily absorbed by the tissue of a patient. In contrast, the more hydrophilic, ionic form may more easily dissolve in body fluids during its placement in the patient. In many cases, longer lasting, more uniform delivery of a bioactive is achieved if an implantable medical device contains more of the non-ionic form than the ionic form.

For example, implantation of a medical device coated with an ionic form of the anesthetic drug bupivacaine (1-butyl-N-(2,6-dimethylphenyl)-2-piperidinecarboxamide hydrochloride) (i.e. Bupivacaine.HCl) may result in the majority of the drug being released into body fluids before the device reaches the desired implantation site.

In one embodiment, the bioactive is bupivacaine. Bupivacaine provided as the water-soluble formulation bupivacaine.HCl, comprising bupivacaine with a proton binding site in the protonated ionic form (2b) below is preferably converted to the formula (2a), with the proton binding site in the deprotonated non-ionic form. Formula (2a) is difficult to dissolve in water, but is more readily soluble in a variety of non-polar solvents. Upon conversion to formula (2a), the piperidine ring of the bupivacaine molecule is deprotonated at the acidic proton binding site. The proton attached to the quaternary amine proton binding site in bupivacaine formula (2b) is an acidic proton with a pKa in water of about 8.09.

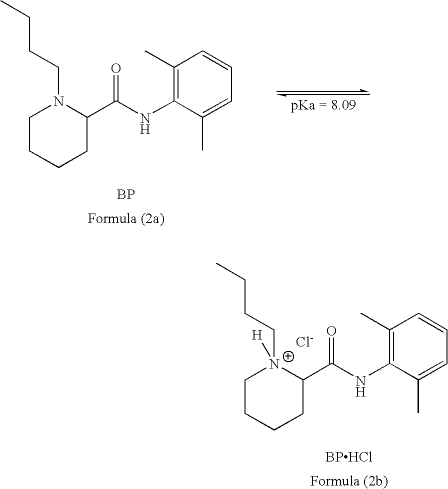

### 1-Butyl-N-(2,6-dimethylphenyl)-2-piperidinecarboxamide

When the bioactive is present with the proton binding site in the non-ionic form, release of the bioactive from the medical device in an aqueous environment occurs over a significantly longer time period than when the bioactive is present in the ionic form. For example, FIG. 7, which is discussed in Example 1, shows a release profile for bupivacaine with the proton binding site in the non-ionic form from a ureteral stent when the stent is placed in solutions having various pH values. As discussed below, increasing the pH from 4.5 to 9.5 slightly slowed the rate of bupivacaine elution from the coating. Release occurs over a period of about 12 days. In comparison, when the medical device contains the ionic form, Bupivacaine.HCl, release occurs under the same conditions within a matter of minutes. Surprisingly, regardless of the pH of the eluting solution, release of the non-ionic form occurs at approximately the same rate. Thus, use of the non-ionic form of a drug for local delivery of the drug offers the potential of a consistent rate of elution of the drug over a range of pH valves in the local environment of the medical device. In addition, a medical device coating consisting of the non-ionic form of bupivacaine positioned against a cellular surface may more easily elute from the device and cross the lipopholic cell membrane than can the ionic form of bupivacaine (for example, Bupivacaine.HCl). Once inside the cell, the non-ionic form will spontaneously convert to the ionic form because the local pH is below the pKa of bupivacaine. The pharmaceutically active form of bupivacaine, which acts by inhibiting sodium ion influx at the Node of Ranvier, is the ionic form.

There are many other bioactives having a proton binding site that may be converted from a non-ionic form that is less soluble in water to a corresponding ionic form that may be substantially more soluble in water. These include lidocaine, mepivacaine and ropovacaine. Other bioactives having a pH-dependent solubility in water or another solvent may be converted to a desired form by adjusting the pH of a coating solution that is contacted with a medical device in a manner effective to form a coating with a desired elution rate. In one embodiment, the bioactive includes a proton binding site having a nitrogen atom. Most preferably, the bioactive is a piperidine-2-carboxanilide comprising the structure of formula (1) above, with R¹ being an alkyl group, R² is an optionally alkyl substituted aryl group and the proton binding site being the tertiary amine bound to moiety R¹. Examples of anesthetic compounds having a proton binding site with an ionic and a non-ionic form are given in Table 1 below.

TABLE 1
Bioactives

| | | | Merck | |
|---|---|---|---|---|
| | | | Index drug | |
| Name | Structure - unprotonated | Structure - protonated | listing | pKa |
| bupivacaine | 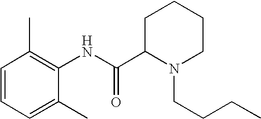 | 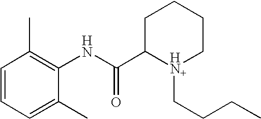 | Neutral HCl | 8.09 |
| lidocaine | 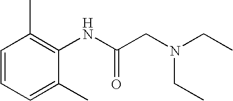 | 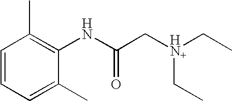 | Neutral HCl | 7.70 |
| mepivacaine | 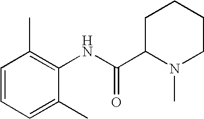 | 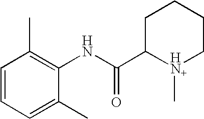 | Neutral HCl | 7.60 |
| ropivacaine | 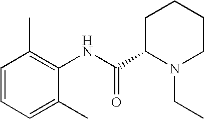 | 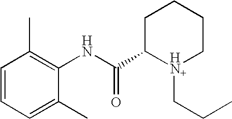 | Neutral HCl | 8.16 |

Other bioactives having a proton binding site include non-anesthetics such as albuterol, amphetamine, beclomethasone, benazepril, bupropion, buspirone, cetirizine, chlordiazepoxide, chlorhexidine cimetidine, clindamycin, clonidine, clorazepate, codeine, dextromethorphan, dicyclomine, diethylpropion, difenoxin, doxorubicin, doxycycline, esmolol, estradiol, fexofenadine, fluoxetine, fluphenazine, hydrocodone, hydromorphone, hydroxyzine, methadone, morphine, methotrexate, minocycline, oxycodone, phenylephrine, propafenone, propoxyphene, pseudoephedrine and verapamil.

In one embodiment, the bioactive is present in a coating containing from about 0.1 μg to about 100 μg of the bioactive per mm² of the gross surface area of the structure. In another embodiment, the coating of bioactive contains from about 1 μg to about 40 μg of the bioactive per mm² of the gross surface area of the structure. “Gross surface area” refers to the area calculated from the gross or overall extent of the structure, and not necessarily to the actual surface area of the particular shape or individual parts of the structure. In other terms, about 100 μg to about 300 μg of bioactive per 0.025 mm of coating thickness may be contained on the device surface.

In certain embodiments of the invention, the thickness of each coating layer is between 0.1 μm and 100 μm. In other embodiments, the thickness of each coating layer is between 0.1 μm and 10 μm. In yet other embodiments, the thickness of each coating layer is between 0.1 μm and 5 μm.

Preparation of the Non-Ionic Form from the Ionic Form

Another aspect provides methods of incorporating a bioactive into a medical device. The bioactive preferably includes a proton binding site providing a pH dependent solubility in a solvent. The methods preferably include forming a solution comprising a bioactive having a proton binding site for an acidic proton of a first pKa in water and contacting the solution with a medical device in a manner effective to incorporate the bioactive into the medical device, for example by forming a coating containing the bioactive on a surface of the medical device and/or by incorporating the bioactive into a base material forming at least a portion of the medical device. The solution preferably has a pH that is controlled to provide a device comprising the bioactive with the proton binding site in a form that is less soluble in water.

The bioactive may be selected to have solubility in an aqueous solvent (e.g., water) that is comparatively higher when the proton binding site is in an ionic form compared to when the proton binding site is in a non-ionic form. Preferably, the proton binding site comprises nitrogen, such as a tertiary amine non-protonated non-ionic form and a quaternary amine cationic protonated form. In one embodiment, the bioactive includes an amine proton binding site providing a pH dependent solubility in water. The solubility of the bioactive in water may be higher when the proton binding site is in a protonated cationic form (e.g., a quaternary amine) than when the proton binding site is in a non-ionic form (e.g., a tertiary amine). Most preferably, the proton binding site is provided as a piperidine or other tertiary amine. The bioactive may be a piperidine carboxanilide, including a bioactive comprising a piperidine-2-carboxanilide of formula (1) above.

The solution may be obtained by dissolving the bioactive with the proton binding site in a solvent at a desired pH. In one embodiment, the bioactive may be provided in a multi-step process. Each proton binding site may bind an acidic proton characterized by a pKa in an aqueous solution depending on the pH of the solution. As described above, when the pH is equal to the pKa in solution, about 50% of the proton binding sites are in the protonated form. Raising the pH of a solution above the pKa increases the proportion of the bioactive with the proton binding site in the unprotonated form compared to the protonated form according to the Henderson-Hasselbach equation above. Decreasing the pH of the first solution below the pKa decreases the proportion of the bioactive with the proton binding site in the unprotonated form and increases the proportion of bioactive molecules with protonated proton binding sites from the first solution. Additional increases in the pH of the solution will result in a greater proportion of the molecule being converted to the non-ionic form (e.g., precipitating out from the first solution).

For certain proton binding sites, such as nitrogen-containing binding sites, the protonated state is a cation and the unprotonated state is neutral or non-ionic. Therefore, increasing the pH of the coating solution (e.g., by adding a base) may decrease the solubility of the bioactive in an aqueous solution, resulting in formation of a solid containing the bioactive with the proton binding site in the unprotonated (non-ionic) form, which can be dissolved in a second solvent to form a coating solution. Therefore, the method may include (1) dissolving the bioactive in a solvent (e.g., water) with the proton binding site in the ionic form to form a first solution; (2) adjusting the pH of the first solution to convert at least a fraction of proton binding sites to the non-ionic form, where the bioactive is less soluble in the solvent when the proton binding site in the non-ionic form than in the ionic form; (3) isolating the fraction of the bioactive with the proton binding site in the non-ionic form from the first solution; (4) dissolving the bioactive with the proton binding site in the non-ionic form in a second solvent to form a coating solution and (5) coating the bioactive in the coating solution onto or into a medical device to incorporate the bioactive having the proton binding site in the non-ionic form.

For example, the bioactive bupivacaine.HCl having a quaternary (protonated) amine cationic proton binding site may be dissolved in water to form a clear first solution at a neutral pH (e.g., about pH 7). The pH of the solution may be lowered until bupivacaine with the proton binding site in the non-ionic deprotonated form precipitates as a white solid out of the first solution. The solid bupivacaine precipate may be isolated from the first solution and dissolved in a suitable organic solvent, such as dichloromethane, and spray coated onto the surface of a medical device to form a bupivacaine coating. The resulting bupivacaine coating may have a reduced rate of elution upon implantation within the urinary tract compared to a coating consisting of bupivacaine HCl.

An aqueous solution of 4-8 g/L of the bupivacaine.HCl may be stirred and its pH raised from about neutral to a pH of about 9.5 to 10 (above the pKa of 8.09 of the acidic proton) by adding a small amount of a strong base, such as NaOH. As the base is added, the water-soluble protonated bupivacaine (formula (1)) is converted to the less soluble deprotonated form of bupivacaine (formula (2)), resulting in precipitation of the bupivacaine as a white solid. The precipitate may be washed and dissolved in an organic solvent as is discussed below.

Optionally, the bioactive may be obtained directly with the proton binding site in a non-ionic form. Although many bioactives, such as bupivacaine, having a proton binding site in a non-ionic and unprotonated form are minimally soluble in water, many may be dissolved in an organic solvent or in a non-polar solvent. Suitable solvents for use with bupivacaine in a non-ionic and unprotonated form include, but are not limited to, for methylene chloride (also known as dichloromethane, DCM), chloroform (CHCl₃), and tetrahydrofuran (THF). Other solvents may be used for the bioactives useful in the present invention include, but are not limited to, alkanes such as pentane, hexane, octane, cyclohexane, heptane, isohexane, butane, pentane, isopentane, 2,2,4-trimethlypentane, nonane, decane, dodecane, hexadecane, eicosane, methylcyclohexane, cis-decahydronaphthalene, N-methylpyrollidone and trans-decahydronaphthalene. Non-polar aromatic solvents may include benzene, toluene, xylene(s), naphthalene, styrene, ethylbenzene, 1-methylnaphthalene, 1,3,5-trimethylbenzene, tetrahydronaphthalene, diphenyl and 1,4-diethylbenzene, among others. Non-polar halohydrocarbon solvents may include chloro-hydrocarbons such as chloroform, methyl chloride, dichloromethane, 1,1-dichloroethylene, ethylene dichloride, ethylidene chloride, propyl chloride, cyclohexyl chloride, 1,1,1-trichloroethane, perchloroethylene, trichloroethylene, butyl chloride, carbon tetrachloride, tetrachloroethylene, chlorobenzene, o-dichlorobenzene, benzyl chloride, trichlorobiphenyl, methylcyclohexane, and 1,1,2,2-tetrachloroethane. Other non-polar solvents may include fluorinated halogenated species such as chlorodiflouoromethane, dichlorofluoromethane, dichlorodifluoromethane, trichlorofluoromethane, 1,2-dichlorotetrafluoroethane, 1,1,2-trichlorotrifluoroethane, perfluoro(methylcyclohexane), and perfluoro-(dimethylcyclohexane).

Other solvents include those with a polar portion, such as alcohols, so long as the solvent molecule itself is not polar, i.e., does not have a dipole moment or dielectric constant near that of water, or higher than that of water. Thus, water is clearly a polar solvent, as is dimethyl formamide (DMF), dimethyl sulfoxide (DMSO), hexamethylphosphoryl amide (HMPA), and hexamethyl phosphoryl triamide (HMPT). However, most organic solvents, such as ethyl acetate, diethyl ether, methyl alcohol, and n-butyl-alcohol, are considerably less polar than water. Organic solvents generally may be used.

In one embodiment, the bioactive with the proton binding site in the non-ionic form is substantially insoluble in water. Intrinsic water solubility for the non-ionic form of the bioactive may be less than about 3%, 2% or 1% by weight, and typically less than about 0.1% or 0.01% by weight. In one example, bupivacaine in its base or non-ionic form has a solubility of about 0.2 mg/mL, while the hydrochloride salt, bupivacaine hydrochloride, is described as being soluble in water. Dexamethasone (Merck and Co., Inc.) is practically insoluble in water, but the disodium phosphate salt is freely soluble in water and is very hygroscopic.

Coating Methods

A bioactive in a solution may be contacted with the medical device in any known manner. For example, a bioactive may be applied by spraying, dipping, pouring, pumping, brushing, wiping, vacuum deposition, vapor deposition, plasma deposition, electrostatic deposition, ultrasonic deposition, epitaxial growth, electrochemical deposition or any other method known to those skilled in the art. The bioactive may be applied to a surface of a base material forming at least a portion of the device as a separate layer or may be included in a layer also including a carrier material. In other embodiments, the bioactive is incorporated into such a base material, for example, by incorporating the bioactive into pores present in a non-metallic base material.

The incorporation of the bioactive into the medical device may be performed by contacting a solution comprising the bioactive and a solvent by any one or more of the following illustrative methods: spray coating, electrostatic deposition (ESD), ultrasonic deposition (USD) and immersion. The spray coating, ESD and USD methods are particularly useful for applying the bioactive to a medical device having a metal base material. The immersion method is particularly useful for applying the coating to a polymeric medical device (e.g., a catheter or plastic stent). However, it will be understood, that the medical device may be coated using any known manner, as well as those mention above.

Spray Coating

The coating material may be dissolved in a suitable solvent(s) and sprayed onto the medical device under a fume hood using a spray gun, such as the Model Number 200 spray gun manufactured by Badger Air-Brush Company, Franklin Park, Ill. 60131. Alignment of the spray gun and medical device may be achieved with the use of laser beams, which may be used as a guide when passing the spray gun up and down the medical device being coated.

Electrostatic Spray Deposition

In another embodiment, the coating material is dissolved in a suitable solvent and then sprayed onto the medical device using an electrostatic spray deposition (ESD) process. The ESD process generally depends on the principle that a charged particle is attracted towards a grounded target. Without being confined to any theory, the typical ESD process may be described as follows:

The solution that is to be deposited on the target is typically charged to several thousand volts (typically negative) and the target held at ground potential. The charge of the solution is generally great enough to cause the solution to jump across an air gap of several inches before landing on the target. As the solution is in transit towards the target, it fans out in a conical pattern which aids in a more uniform coating. In addition to the conical spray shape, the electrons are further attracted towards the conducting portions of the target, rather than towards the non-conductive base the target is mounted on, leaving the coating mainly on the target only.

In the ESD process, the coating solution is forced through a capillary, which is subjected to an electrical field. The solvent mixture leaves the capillary in the form of a fine spray, the shape of which is determined by the electrical field. The medical device is then coated by placing it in the spray and allowing the solvent to evaporate, leaving the desired coating on the surface of the device.

The ESD method allows for control of the coating composition and surface morphology of the deposited coating. In particular, the morphology of the deposited coating may be controlled by appropriate selection of the ESD parameters, as set forth in WO 03/006180 (Electrostatic Spray Deposition (ESD) of biocompatible coatings on Metallic Substrates), the contents of which are incorporated by reference. For example, a coating having a uniform thickness and grain size, as well as a smooth surface, may be obtained by controlling deposition conditions such as deposition temperature, spraying rate, precursor solution, and bias voltage between the spray nozzle and the medical device being coated. The deposition of porous coatings is also possible with the ESD method.

Ultrasonic Spray Deposition

In another embodiment, the medical device is coated using an ultrasonic spray deposition (USD) process. Ultrasonic nozzles employ high frequency sound waves generated by piezoelectric transducers which convert electrical energy into mechanical energy. The transducers receive a high frequency electrical input and convert this into vibratory motion at the same frequency. This motion is amplified to increase the vibration amplitude at an atomizing surface.

The ultrasonic nozzle is configured such that excitation of the piezoelectric crystals creates a longitudinal standing wave along the length of the nozzle. The ultrasonic energy originating from the transducers undergoes a step transition and amplification as the standing wave traverses the length of the nozzle. The nozzle is designed such that a nodal plane is located between the transducers. For ultrasonic energy to be effective for atomization, the nozzle tip must be located at an anti-node, where the vibration amplitude is greatest. To accomplish this, the nozzle's length must be a multiple of a half-wavelength. In general, high frequency nozzles are smaller, create smaller drops, and consequently have smaller maximum flow capacity than nozzles that operate at lower frequencies.

Liquid introduced onto the atomizing surface absorbs some of the vibrational energy, setting up wave motion in the liquid on the surface. For the liquid to atomize, the vibrational amplitude of the atomizing surface must be carefully controlled. Below a critical amplitude, the energy is insufficient to produce atomized drops. If the amplitude is excessively high, cavitation occurs. Only within a narrow band of input power is the amplitude ideal for producing the nozzle's characteristic fine, low velocity mist. Since the atomization mechanism relies only on liquid being introduced onto the atomizing surface, the rate at which liquid is atomized depends solely on the rate at which it is delivered to the surface.

For example, the medical device, such as a stent, is coated using an ultrasonic spray nozzle, such as those available from Sono-Tek Corp., Milton, N.Y 12547. The solution is loaded into a 10.0 mL syringe, which is mounted onto a syringe pump and connected to a tube that carries the solution to the ultrasonic nozzle. The syringe pump is then used to purge the air from the solution line and prime the line and spay nozzle with the solution. The stent is loaded onto a stainless steel mandrel in the ultrasonic coating chamber by the following method. The stent is held on a mandrel by silicone tubing at each end.

In one embodiment, the stent is 6 mm in diameter and the mandrel's diameter is 4.12 mm. In another embodiment, the stent is 8 mm in diameter and the mandrel's diameter is 6.51 mm. In yet another embodiment, the stent is 10 mm in diameter and the mandrel's diameter is 4.97 mm. In another embodiment, a mandrel of 3.52 mm diameter is used for all of the above stents. Other sized stents and mandrels may also be used in the present invention. The mandrel is fastened onto a motor, positioned below the ultrasonic nozzle. The motor rotates the mandrel at a pre-set speed and translationally moves it so that all parts of the stent pass underneath the ultrasonic spray. In one embodiment, the rotational speed is set to 10 rpm and the translational speed is set to 0.01 mm per second. In another embodiment, the rotational speed is set to 60 rpm and the translational speed is set to 0.05 mm per second. In yet another embodiment, the rotational speed is set to 110 rpm and the translational speed is set to 0.19 mm per second. Other speeds and combinations may also be used in the present invention. The coating chamber is purged with nitrogen to displace oxygen in the system. During the process, the stent is kept at ambient temperature and in a closed chamber.

In one embodiment of the invention, the oxygen concentration is reduced to between 0 and 2000 ppm. In another embodiment the oxygen concentration is reduced to between 0 and 500 ppm. In yet another embodiment the oxygen concentration is reduced to between 0 and 100 ppm.

Immersion

In another embodiment, the medical device is immersed into the solution comprising the bioactive at a desired pH until the desired amount of bioactive is absorbed by the medical device. Upon removal from the solution, as the solution material dries and solidifies on the medical device, it forms a coating. Methods for dip coating a medical device are disclosed in, for example, U.S. Pat. No. 6,153,252, the contents of which are incorporated by reference.

In one example, ureteral stents are coated by sequentially dipping them into a solution of a bioactive, drying, and then dipping again. In another example, polyurethane ureteral stents are coated by sequentially dipping them into a methanol solution of a bupivacaine bioactive, drying, and then dipping them into a solution of a second bioactive or a carrier material, and then dried. The dipping process may be repeated as many times as desired until the desired coating depth is achieved. A lyophilizing process, with unitary or sequential cycles of applying the bioactive and then freeze-drying and applying a vacuum, may also be used to remove the solvent and consolidate the coating.

Medical Devices Including the Non-Ionic Form of a Bioactive

Preferably, the medical device is configured for implantation within a body vessel. In various embodiments, the medical devices may be placed in the coronary vasculature, esophagus, trachea, colon, bladder, heart, vessels, lumens, intestines, biliary tract, urinary tract, prostate, or brain, for example. Medical devices may include, but are not limited to, stents, stent grafts, shunts, vascular grafts, catheters, cannulas, guide wires, catheter balloons, filters (e.g. vena cava filters), sutures, staples, anastomosis devices, vertebral disks, bone pins, suture anchors, hemostatic barriers, clamps, screws, plates, clips, vascular implants, tissue adhesives and sealants, tissue scaffolds, valves (e.g. venous valves), abdominal aortic aneurysm (AAA) grafts, embolic coils, various types of dressings, bone substitutes, intraluminal devices, vascular supports, biliary stents or other known bio-compatible devices.

In one embodiment, the medical device comprises a coated drainage device, such as a catheter or drainage stent. The medical device may be adapted to provide temporary internal drainage from the ureteropelvic junction to the bladder, such as the SOF-FLEX stent (Cook, Inc, Bloomington, Ind.). The stent may be a metallic frame or woven tubular stent, such as a self-expanding or balloon-expandable stent. The medical device may be a bifurcated stent, a coronary vascular stent, a urethral stent, a ureteral stent, a biliary stent, a tracheal stent, a gastrointestinal stent, or an esophageal stent, for example. More specifically, the stent may be, for example, a Wallstent variety stent or a Gianturco-Roubin, Palmaz-Shatz, Wiktor, Strecker, Cordis, AVE Micro Stent, Igaki-Tamai, Millenium Stent, Steeplechaser stent (Johnson & Johnson), Cypher (Johnson & Johnson), Sonic (Johnson & Johnson), BX Velocity (Johnson & Johnson), Flexmaster (JOMED) JoStent (JOMED), S7 Driver (Medtronic), R-Stent (Orbus), Technic stent (Sorin Biomedica), BiodivYsio (Abbott Laboratories), DuraFlex (Avantec Vascular), NIR stent (Boston Scientific), Express 2 stent (Boston Scientific), Liberte stent (Boston Scientific), Achieve (Cook/Guidant), S-Stent (Guidant), Vision (Guidant), or Multi-Link (Guidant). Some exemplary stents are disclosed in U.S. Pat. Nos. 5,292,331 to Boneau, 6,090,127 to Globerman, 5,133,732 to Wiktor, and 4,739,762 to Palmaz, 5,421,955 to Lau. Desirably, the stent is a vascular stent such as the commercially available Gianturco-Roubin FLEX-STENT®, GRII™, SUPRA-G, or V FLEX coronary stents from Cook Incorporated (Bloomington, Ind.).

In another embodiment, the medical device comprises a plastic biliary stent. Such a stent may be coated with ciprofloxacin or a methyl or ethyl derivative. Such stents are typically about 10 to about 60 mm in length and designed to expand to a diameter of about 2 to about 6 mm when inserted into the vascular system of the patient. The Gianturco-Roubin stent in particular is typically about 12 mm to about 25 mm in length and designed to expand to a diameter of about 2 mm to about 4 mm when so inserted.

These stent dimensions are, of course, applicable to exemplary stents employed in the coronary arteries. Structures such as stents or catheter portions intended to be employed at other sites in the patient, such as in the aorta, esophagus, trachea, colon, biliary tract, or urinary tract will have different dimensions more suited to such use. For example, aortic, esophageal, tracheal and colonic stents may have diameters up to about 25 mm and lengths about 140 mm or longer.

In another embodiment, the medical device is a graft. A graft may be a patch made from, for example, Dacron® polyester or other artificial material, or may be a lyophilized laminate made from porcine SIS (small intestine submucosa) and available from Cook Biotech, Inc., West Lafayette, Ind. Other medical devices include polymeric or elastomeric ureteral stents or urethral catheters. These devices are typically made from a silicone elastomer, urethane, Thoralon® elastomer, or other polymeric material. In one embodiment, the bioactive is coated onto the device or substrate, such as the outer surface of a ureteral stent or urinary catheter. Alternatively, the drug may be coated onto the struts of a Zilver or coronary stent, or onto the fabric of a stent-graft.

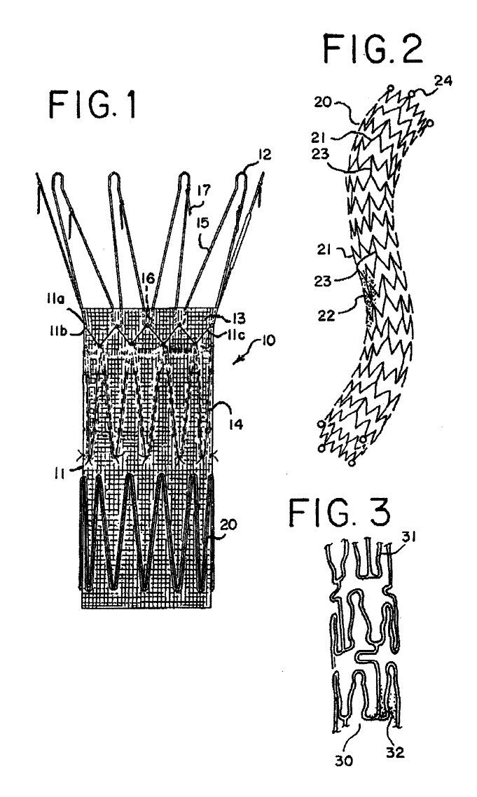

FIGS. 1-5a depict several exemplary medical devices which may serve as substrates for coatings of the non-ionic form of the bioactive. FIG. 1 depicts a stent-graft 10 which may be used for repair of an arterial aneurysm. Stent-graft 10 includes graft 11, anchoring stent 12, internal stent 14 and external stent 20. The stents are formed with a combination of struts 15 and apices 16. Barbs 17 may be used to anchor the stent to the body lumen in which the stent is placed. The stents are secured to graft 11 with stitches 11a, 11b and 11c. A coating 13 may be placed on the graft, or may also be placed on the stents themselves.

FIG. 2 depicts a biliary stent with a coating. Biliary stent 20 is a hollow metal structure formed with struts 21 and apices 23 in the form of a Z, the rows of Zs joined periodically to ensure axial flexibility while also imparting radial strength. Stent 20 may include markers 24 made of gold or other radiopaque material. Coating 22, which may include the non-ionic form of a bioactive, may be placed on the struts and apices that form the stent.

FIG. 3 depicts a coronary or vascular stent 30. Coronary stent 30 is typically a hollow metal structure made from Nitinol or other shape memory alloy and heat treated so that it returns to an expanded shape upon reaching body temperature. The stent includes struts 31 and apices 33 and a coating 32 may be placed on the struts and apices that form the stent. Again, the coating 32, may include the non-ionic form of a bioactive.

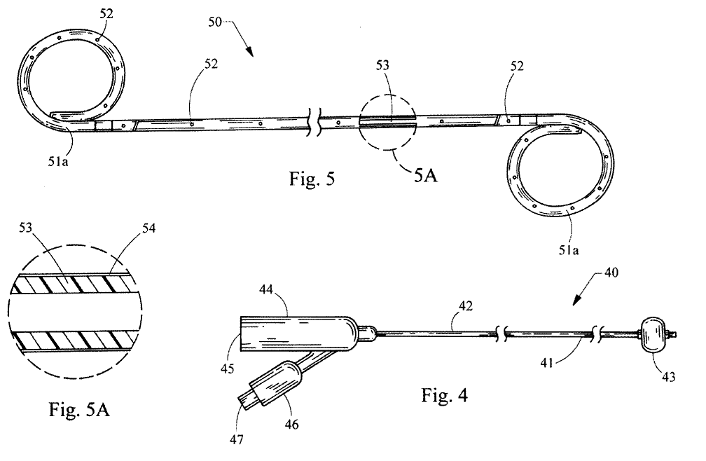

FIG. 4 depicts a Foley catheter. A Foley catheter may be implanted for weeks at a time, and may be coated to resist infection or encrustation. Foley catheter 40 includes drainage shaft 41 with coating 42. An expansion balloon 43 is placed in the bladder to retain the Foley. The Foley catheter may also include a drainage connector 44 on its proximal end 45, and includes an inflation connector 46 for a balloon inflation lumen 47. The expansion balloon 43 may also include a coating 42. Coating 42 may include the non-ionic form of a bioactive.

A pigtail-type ureteral stent is depicted in FIGS. 5 and 5a. Ureteral stent 50 includes distal and proximal ends 51a and intermediate length 53. Orifices 52 allow urine to drain from the kidney into the stent. A portion of the stent may be coated with a coating 54 of a bioactive.

Composition of the Medical Device

A variety of conventional materials can be employed as the base material of the medical device. The medical device may be formed from a biocompatible base material, which may be either elastic or inelastic. The base material may be either biodegradable or non-biodegradable, and a variety of biodegradable polymers are known. Moreover, some bioactives have sufficient strength to serve as the base material of some useful structures.

The materials used in the medical device may be selected from a well-known list of suitable metals and polymeric materials appropriate for the particular application, depending on necessary characteristics that are required (self-expansion, high radial force, collapsibility, etc.). Suitable metals or metal alloys include stainless steels (e.g., 316, 316L or 304), nickel-titanium alloys including shape memory or superelastic types (e.g., nitinol or elastinite); inconel; noble metals including copper, silver, gold, platinum, paladium and iridium; refractory metals including Molybdenum, Tungsten, Tantalum, Titanium, Rhenium, or Niobium; stainless steels alloyed with noble and/or refractory metals; magnesium; amorphous metals; plastically deformable metals (e.g., tantalum); nickel-based alloys (e.g., including platinum, gold and/or tantalum alloys); iron-based alloys (e.g., including platinum, gold and/or tantalum alloys); cobalt-based alloys (e.g., including platinum, gold and/or tantalum alloys); cobalt-chrome alloys (e.g., elgiloy); cobalt-chromium-nickel alloys (e.g., phynox); alloys of cobalt, nickel, chromium and molybdenum (e.g., MP35N or MP20N); cobalt-chromium-vanadium alloys; cobalt-chromium-tungsten alloys; platinum-iridium alloys; platinum-tungsten alloys; magnesium alloys; titanium alloys (e.g., TiC, TiN); tantalum alloys (e.g., TaC, TaN); L605; magnetic ferrite; bioabsorbable materials, including magnesium; or other biocompatible metals and/or alloys thereof.

In some embodiments, the medical device itself, or any portion of a frame included in the medical device, can comprise one or more metallic bioabsorbable materials. Suitable metallic bioabsorbable materials include magnesium, titanium, zirconium, niobium, tantalum, zinc and silicon and mixtures and alloys. For example, a zinc-titanium alloy such as discussed in U.S. Pat. No. 6,287,332, which is incorporated herein by reference in its entirety, can be used. The metallic bioabsorbable material can further contain lithium, sodium, potassium, calcium, iron and manganese or mixtures thereof. For example, an alloy containing lithium:magnesium or sodium:magnesium can be used. The physical properties of the device or device frame can be controlled by the selection of the metallic bioabsorbable material, or by forming alloys of two or more metallic bioabsorbable materials. For example, when 0.1% to 1%, percentage by weight, titanium is added to zinc, the brittle quality of crystalline zinc can be reduced. In another embodiment, when 0.1% to 2%, percentage by weight, gold is added to a zinc-titanium alloy, the grain size of the material is reduced upon cures and further and the tensile strength of the material increases.

The medical device may be formed through various methods, such as welding, laser cutting, or molding, or it may consist of filaments or fibers that are wound or braided together to form a continuous structure. The medical device may be a grafted stent in which the bioactive is incorporated into the graft material.

In some embodiments, where the structure of the medical device is composed of a radiolucent material such as polypropylene, polyethylene, or others above, a conventional radiopaque coating may and preferably should be applied to it. For example, where the medical device is a vascular stent, the radiopaque coating provides a means for identifying the location of the stent by X-ray or fluoroscopy during or after its introduction into the patient's vascular system.

In one embodiment, a bioactive is placed directly on the surface of the medical device (or on a primer layer, which is placed on the surface of the medical device) and forms the outermost coating layer on the medical device. In another embodiment one or more barrier layers are placed over at least a portion of the bioactive. In certain embodiments, the primer layer may enhance the adhesion of the bioactive, or a layer containing the bioactive, to the medical device.

A primer layer may be added to the surface of the medical device before the deposition of additional coating layers. Such a layer may promote adhesion of further coating layers to the device surface. The primer layer may comprise, for example, parylene or a parylene derivative, silane, acrylate polymer/copolymer, acrylate carboxyl and/or hydroxyl copolymer, polyvinylpyrrolidone/vinylacetate copolymer (PVP/VA), olefin acrylic acid copolymer, ethylene acrylic acid copolymer, epoxy polymer, polyethylene glycol, polyethylene oxide, polyvinylpyridine copolymers, polyamide polymers/copolymers, polyimide polymers/copolymers, ethylene vinylacetate copolymer and/or polyether sulfones.

In other embodiments, the bioactive forms part of the base structure of the medical device itself. Alternatively, the medical device may have holes, wells, slots, grooves, or the like for containing the bioactive, possibly covered by a barrier layer, and/or mixtures of carrier material/bioactive, possibly separated by barrier layers. Illustrative medical devices are disclosed in U.S. Publication No. 2004/0243225A1, published Dec. 2, 2004, the contents of which are incorporated by reference.

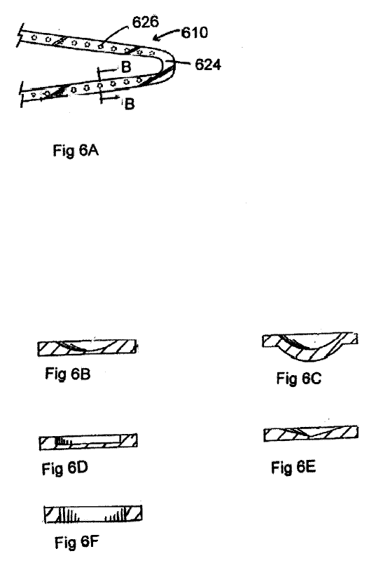

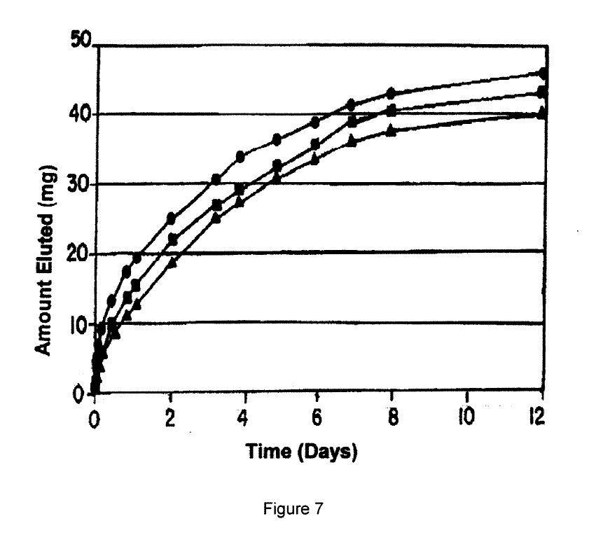

Referring to FIG. 6A, a surface 624 of a frame portion 610 of a medical device frame comprises a plurality of holes or wells 626 containing a bioactive. The well 626 may contain a coating comprising the bioactive alone, a mixture of the bioactive with a suitable carrier material or a coating comprising multiple layers, including, for example, one or more barrier layers. FIGS. 6B-6F show cross sectional views of various well or hole configurations along line B-B′ of frame 610. The holes or wells may have any suitable shape or size, including a concave well formed by removing a portion of the frame 610 (FIG. 6B) or formed by re-shaping a portion of the frame (FIG. 6C), a square shaped well (FIG. 6D), a V-shape well (FIG. 6E), or a hole (FIG. 6F). The holes or wells and the like, described above, may be formed in the surface of the medical device by any suitable technique. For example, such techniques include drilling or cutting by utilizing lasers, electron-beam machining and the like or employing photoresist procedures and etching the desired apertures.

In certain embodiments, the bioactive may be incorporated into a biodegradable medical device, or the bioactive may be incorporated into a medical device in any other known manner. A medical device containing a bioactive therein may also include a layer including a bioactive, a barrier layer, a layer containing both a bioactive and a carrier material, or any combination of these, including combinations including multiple layers.

Optionally, the bioactive is mixed with a carrier material and this mixture applied to the medical device (or on a primer layer, which is placed on the surface of the medical device). Alternatively, the carrier material may be applied to the surface of the medical device, or the primer layer, and the bioactive absorbed into the carrier material. In such configurations, the release of the bioactive may be dependent on factors including the composition, structure and thickness of the carrier material. In one embodiment, the carrier material may contain pre-existing channels, through which the bioactive may diffuse, or channels created by the release of the bioactive, or another soluble substance, from the carrier material. In other embodiments, a combination of one or more layers of primer, bioactive, mixtures of carrier material/bioactive, and barrier layers are present. For example, the bioactive may be mixed with a carrier material and coated onto the medical device and then over coated with one or more barrier layer(s). In yet other embodiments, multiple layers of bioactive, or mixtures of carrier material/bioactive, possibly separated by barrier layers, are present to form a multicoated medical device. In certain embodiments, different bioactives are present in the different layers.

Desirably, a polymer coating component adequately adheres to the surface of the medical device, and deforms readily after it is adhered to the device. The molecular weight of the polymer(s) should be high enough to provide sufficient toughness so that the polymers will not be rubbed off during sterilization, handling, or deployment of the medical device and will not crack when the device is expanded. The molecular weight of the polymer may be varied to influence the rate of release of the bioactive. For example, to obtain a slower rate of release, a polymer(s) of higher molecular weight may be used.

Surface Preparation, Optional Layers, and Layer Thickness

In embodiments where the bioactive is coated onto a surface of the medical device, it may be advantageous to prepare the surface of the medical device before depositing a coating thereon. Useful methods of surface preparation can include, but are not limited to cleaning; physical modifications such as etching, drilling, cutting, or abrasion; and chemical modifications such as solvent treatment, the application of primer coatings, the application of surfactants, plasma treatment, ion bombardment, covalent bonding and electrochemical methods such as electropolishing, striking, electroplating and electrochemical deposition. Such surface preparation may serve to activate the surface and promote the deposition or adhesion of the coating on the surface. Surface preparation can also selectively alter the release rate of the bioactive.

Any additional coating layers can similarly be processed to promote the deposition or adhesion of another layer, to further control the release of the bioactive, or to otherwise improve the biocompatibility of the surface of the layers. For example, plasma treating an additional coating layer before depositing a bioactive thereon may improve the adhesion of the bioactive, increase the amount of bioactive that can be deposited, and allow the bioactive to be deposited in a more uniform layer.

A more complete understanding of the present invention can be obtained by reference to the following specific Examples. The Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitations. Modifications and variations of the invention as herein before set forth can be made without departing from the spirit and scope thereof, and, therefore, only such limitations should be imposed as are indicated by the appended claims.

### EXAMPLE 1

### Preparation of Ureteral Stents Containing Bupivacaine and Elution from the Stents in an Aqueous Medium

An approximately 0.6% solution of bupivacaine having a tertiary amine in the non-ionic form (formula (2a) above) in dichloromethane was loaded into a syringe mounted onto a syringe pump and connected to a tube that carried the solution to an ultrasonic nozzle (Model 06-05108—Sono-Tek Co., Poughkeepsie, N.Y. 12547.) A nozzle was 6 mm diameter, 11 mm long and used with a nominal frequency of 120 kHz. Air was purged from the solution line and the spray nozzle primed with the solution. The coating chamber was purged with nitrogen to attain an oxygen concentration below 100 ppm. The solution was sprayed onto ureteral stent substrates 20 to 280 mm long (polyurethane double pigtail soflex ureteral stent, Cook Incorporated (Bloomington, Ind.)). A smooth, uniform coating was observed. Control stents were coated with the same concentration of Bupivacaine.HCl using the above coating method.

The stents were placed in solutions at different pH values to test the elution of bupivacaine over time. FIG. 7 shows the elution of bupivacaine at pH values of 4.5, 7.0 and 9.5 over a 12-day period. Acetic acid/sodium acetate was used for the pH 4.5 buffer, 0.1 M phosphate-buffered solution (PBS) for the pH 7.0 buffer, and sodium carbonate for the pH 9.5 buffer. In comparison, stents coated with bupivacaine.HCl eluted fully within minutes in an aqueous environment.

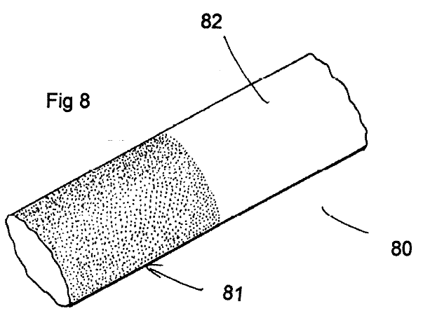

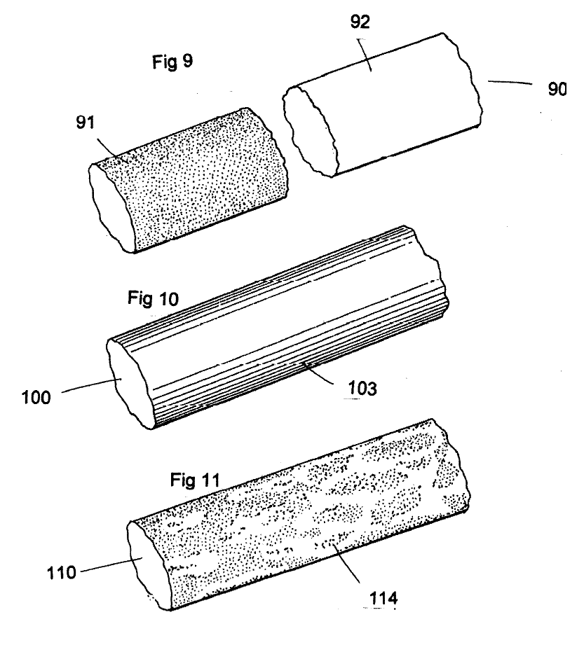

FIGS. 8-11 depict photomicrographs of the coatings showing the uniformity and tenacity of the non-ionic coatings, in contrast to the water-soluble ionic coatings. FIG. 8 depicts a ureteral stent 80, with left portion 81 coated with bupivacaine.HCl; the portion on the right 82 was identically coated but was dipped in water for one minute and the coating is substantially dissolved. FIG. 9 depicts a relatively rough, crystalline coating 91 of bupivacaine.HCl on a ureteral stent 90, on the left, and the same stent after a one-minute ex vivo exposure to a pig ureter. Most of the coating 92 has been dissolved.

In contrast, FIG. 10 depicts another stent 100 coated with non-ionic bupivacaine 103 that has been exposed to an aqueous environment for several days, with virtually no effect on the coating, because as is well known, the non-ionic, neutral form of bupivacaine is only very slightly soluble in water. FIG. 11 then depicts the same type of stent 110 with a coating 114 of the non-ionic form of bupivacaine after 24 hours in an ex vivo test with a pig ureter. In contrast to the ionic form of the coating, most of the non-ionic form of the bupivacaine coating remains on the stent after 24 hours. This shows the durability of the coating and its resistance to dissolution or erosion by body fluids.

### EXAMPLE 2

### Immersion of Polymer Stent in a Solution of Bupivacaine and Methanol

The pH of a typical coating solution used to coat a polyurethane drainage stent was measured. The pH of 1 L of methanol was measured at about 7.00. A polyurethane ureteral stent (SOF-FLEX, Cook Incorporated, Bloomington, Ind.) having a 6-French outer diameter, an inner diameter of 2 mm and a length of about 34 cm was placed in the methanol. After 1 hour, the pH was measured at about 6.35. A total of 235 g of bupivacaine base (formula (2a) above) was dissolved in the 1 L of methanol containing the ureteral stent. After 1 hour, the pH of the bupivacaine methanol solution in the presence of the ureteral stent was measured at about 10.00. Given the high pH, it is believed that most of the bupivacaine in the solution and in contact with the ureteral stent in the solution is in the non-protonated, non-ionic form that is less soluble in water.

### EXAMPLE 3

### Bupivacaine Coatings Applied to Polymer Stents by Immersion at High and Low Doses

A series of eight polyurethane stents described in Example 2 were coated with bupivacaine by placing the stents in a coating solution containing methanol and bupivacaine. Four high dose coated stents were formed by placing the uncoated stents in a first solution containing 470 g bupivacaine base (100% saturation) in 1 liter of methanol for 100 minutes. Four low dose coated stents were formed by placing the uncoated stents in a second solution containing 235 g bupivacaine base in 1 liter of methanol (50% saturation) for 60 minutes. “Bupivacaine base” refers to bupivacaine obtained according to formula (2a) above (i.e., with the proton binding site in the unprotonated, non-ionic form).

The stents were removed, dried and the amount of bupivacaine retained on each stent was determined by immersing the stent in an aqueous dibasic buffer at a pH of 7.1 (0.1 M sodium phosphate) for a period of 24 hours, removing the stent and measuring the ultraviolet absorption of the aqueous dibasic buffer solution at 262 nm (using beer-lambert molar extinction coefficient of 427 l/mol cm, a 1 cm cell length, a volume of 50 mL per sample), and correlating the UV absorption of the aqueous buffered solution to the amount of bupivacaine eluted from the stent over 24 hours in the sample. The stents were then transferred to another buffered solution and the UV measurement was repeated to determine the amount of bupivacaine eluting from the stent in the second 24 hour period. The “optical dose” of bupivacaine absorbed by each stent during immersion was calculated by UV detection and Beer-Lambert correlation of the absorbance due to bupivacaine that eluted from the stent into the buffered solution, and is shown in Table 2 for the low dose stents and Table 3 for the high dose stents. The “gravimetric dose” was determined by weighing the stents before immersion and after drying.

TABLE 2
Mass Balances, 50% Saturation, 60 min
Stent 50/60 Mass Balance

| | Grav. Dose | Optical Dose | Mass |
|---|---|---|---|
| Article | BP (mg) | BP (mg) | Balance |
| 5 | 64.84 | 68.61 | 105.81% |
| 10 | 64.61 | 68.94 | 106.70% |
| 19 | 62.40 | 67.91 | 108.83% |
| 20 | 63.58 | 68.84 | 108.27% |

TABLE 3
Mass Balances, 100% Saturation, 100 min
Stent 100/100 Mass Balance

| | Grav. Dose | Optical Dose | Mass |
|---|---|---|---|
| article | BP (mg) | BP (mg) | Balance |
| 25 | 160.48 | 166.96 | 104.04% |
| 27 | 162.32 | 172.41 | 106.22% |
| 37 | 154.47 | 161.92 | 104.82% |
| 39 | 157.87 | 165.68 | 104.95% |

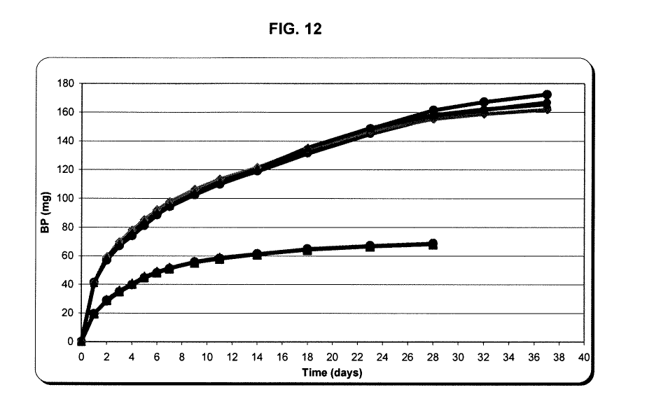

The amount of bupivacaine that eluted from each of the eight stents over a period of 28 days (low dose) and 38 days (high dose) into a buffered aqueous solution at a pH of about 7 is shown in FIG. 12. The four high dose samples provided a first group of elution curves 100 showing elution of about 180 mg of bupivacaine within about 38 days; the four low dose samples provided a second group of elution curves 200 showing elution of up to about 60 mg of bupivacaine in about 28 days.

We claim:

1. A medical device comprising:
a base material and a bioactive in contact with the base material, the bioactive having a proton binding site with a non-ionic form and an ionic form, the bioactive being less soluble in water when the proton binding site is in the non-ionic form than when the proton binding site is in the ionic form, wherein at least 5% w/w of the bioactive is present with the proton binding site in the non-ionic form and wherein the bioactive is not an anesthetic.

2. The medical device of claim 1, wherein the proton binding site has a protonated ionic form and an unprotonated non-ionic form.

3. The medical device of claim 2, wherein the proton binding site includes a nitrogen atom.

4. The medical device of claim 3, wherein the proton binding site is a tertiary amine in the in the non-ionic form and a cationic quaternary amine in the ionic form.

5. The medical device of claim 1, wherein the bioactive comprises a chemical structure of the formula: 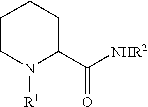
where R¹ is alkyl and R² is an optionally alkyl substituted phenyl.

6. The medical device of claim 1, wherein the base material comprises a polymer.

7. The medical device of claim 1, wherein the bioactive is present in a layer on a surface of the base material.

8. The medical device of claim 7, wherein the layer further comprises one or more materials selected from the group consisting of a bioelastomer, PLGA, PLA, PEG, a hydrogel, microcrystalline cellulose, hydroxypropylmethyl cellulose, hydroxypropyl cellulose, cellulose products, cellulose derivatives, polysaccharides, and polysaccharide derivatives, lactose, dextrose, mannitol, derivatives of lactose, dextrose, mannitol, starch and starch derivatives.

9. The medical device of claim 6, wherein the bioactive is present within pores contained within the base material.

10. A medical device comprising:
a base material and an anesthetic in contact with the base material, the anesthetic having a proton binding site with a non-ionic form and an ionic form, the anesthetic being less soluble in water when the proton binding site is in the non-ionic form than when the proton binding site is in the ionic form, wherein at least 5% w/w of the anesthetic is present with the proton binding site in the non-ionic form and wherein the medical device is not a ureteral stent.

11. The medical device of claim 10, the anesthetic having a amine proton binding site for an acidic proton with a pKa, the device being formed by contacting the base material with a solution including the anesthetic in a solvent at a pH above the pKa of the acidic proton and removing the solvent to form the medical device comprising the anesthetic.

12. The medical device of claim 10, wherein the anesthetic comprises a chemical structure of the formula: 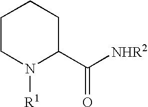
where the N bonded to R¹ is the proton binding site, R¹ is alkyl and
R² is an optionally alkyl substituted phenyl.

13. The medical device of claim 12, wherein R¹ is methyl, n-propyl or n-butyl and R² is 2,6-dimethylphenyl.

14. The medical device of claim 12, where the anesthetic absorbs light at a characteristic peak at 262 nm.

15. The medical device of claim 12, where the medical device comprises polyurethane.

16. A method for incorporating a bioactive into a medical device, the method comprising:
a. dissolving a bioactive in a solvent to form a solution, the bioactive having a proton binding site for an acidic proton having a first pKa and having an ionic form and a non-ionic form in aqueous solution, the bioactive being less soluble in water when the proton binding site is in the non-ionic form than when the proton binding site is in the ionic form,
b. maintaining the pH of the solution above the pKa of the acidic proton such that the solution contains the bioactive with the proton binding site in the non-ionic form; and
c. contacting the medical device with the solution in a manner effective to incorporate the bioactive with the proton binding site in the non-ionic form into the medical device.

17. The method of claim 15, where the ionic form of the proton binding site is protonated and the non-ionic form of the proton binding site is deprotonated.

18. The method of claim 16, wherein the bioactive comprises a chemical structure of the formula: 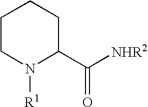
where the N bonded to R¹ is the proton binding site, R¹ is alkyl and
R² is an optionally alkyl substituted phenyl.

19. A method of manufacturing a medical device incorporating a bioactive, the method comprising:
a. dissolving a bioactive having a proton binding site in a solvent to form a solution, the bioactive having a greater solubility in water when the proton binding site is in an ionic form than a non-ionic form; and
b. contacting a surface of the medical device with the solution in a manner effective to incorporate the bioactive with the proton binding site in the non-ionic form into the medical device.

20. The method of claim 19, further comprising the steps of:
c. dissolving the bioactive with the proton binding site in the ionic form in a first solvent to form a pre-coating solution having a first pH;
d. lowering the pH of the pre-coating solution to convert at least a portion of the bioactive proton binding site from the ionic form to the non-ionic form; and
e. isolating at least a portion of the bioactive having the proton binding site in the non-ionic form from the pre-coating solution;
f. dissolving the portion of the bioactive isolated in step (e) in the solvent to form the solution.
